(19) United States
(12) Patent Application Publication
(10) Pub. No.: US 2004/0034045 A1
(43) Pub. Date: Feb. 19, 2004

(54) Inhibitors of thrombin induced platelet aggregation

(75) Inventor: Fatih M. Uckun, White Bear Lake, MN (US)

Correspondence Address: MERCHANT & GOULD PC, P.O. BOX 2903, MINNEAPOLIS, MN, 55402-0903, US

(73) Assignee: Parker Hughes Institute, Roseville, MN (US)

(21) Appl. No.: 10/449,408

(22) Filed: May 29, 2003

Related U.S. Application Data
(63) Continuation of application No. PCT/US01/02195, filed on Jan. 23, 2001.

(30) Foreign Application Priority Data
Nov. 29, 2000 (WO) PCT/US00/42345

(51) Int. Cl. A61K 31/517

(52) U.S. Cl. 514/266.3; 514/266.4

(57) ABSTRACT

The present invention describes a therapeutic method useful for treating or preventing a condition of platelet aggregation in a subject including administering a pharmaceutically effective amount of a compound or composition that inhibits JAK-3 and/or tyrosine phosphorylation of STAT-3 and inhibits thrombin induced platelet aggregation. The condition of platelet aggregation includes hematopoietic and cerbrovascular diseases.

This application is being filed as a PCT International Patent application in the name of Parker Hughes Institute, a U.S. national corporation, (applicant for all countries except US), and Fatih M. Uckun, a U.S. citizen (applicant for US only), on Jan, 23, 2001, designating all countries.

### FIELD OF THE INVENTION

The present invention relates to a therapeutic method for treating or preventing a disease or condition of platelet aggregation in a subject wherein the method includes administering a pharmaceutically effective amount of a compound that inhibits platelet aggregation and specifically, thrombin induced platelet aggregation.

### BACKGROUND OF THE INVENTION

Heart disease, a common cause of death in today's society, is often a result of ischemic syndromes that are produced by atherosclerosis and arteriosclerosis including myocardial infarction, chronic unstable angina, transient ischemic attacks and strokes, peripheral vascular disease, arterial thrombosis, preeclampsia, embolism, restenosis and/or thrombosis following angioplasty, carotid endarterectomy, anastomosis of vascular grafts, and other cardiovascular devices. These syndromes represent a variety of stenotic and occlusive vacular disorders thought to be initiated by platelet aggregation on vessel walls or within the lumen by blood-born mediators thereby forming thrombin that restrict blood flow.

The basic mechanism of platelet aggregation has been well studied. The mechanism starts with a blood vessel injury such as narrowing of the lumen, plaque formation, and the presence of foreign bodies/medical instruments. This injury leads to platelet activation and binding of fibrinogen and ligands. Upon ligand binding, the JAK (Janus-family Kinase) kinases, a family of cytoplasmic protein tyrosine kinases which mediate cytokine receptor signaling, undergo tyrosine phosphorylation and activate the cytoplasmic latent forms of the STAT family transcription factors (Signal Transducers and Activators of Transcription). In an investigation of platelet aggregation in mice deficient in JAK-3, which maps to human chromosome 19p12-13.1, a decrease in thrombin-induced platelet aggregation was discovered by the Applicant.

Gelotte, U.S. Pat. No. 5,972,967 and Scarborough, et al. U.S. Pat. No. 5,968,902 have described certain compounds and compositions that inhibit binding to a platelet by limiting the binding of fibrinogen. Nevertheless, there still is a need for finding compounds and improved methods to treat or prevent a condition of platelet aggregation.

### SUMMARY OF THE INVENTION

The present invention, as embodied and broadly described herein, relates to a therapeutic method for treating or preventing a disease or condition of platelet aggregation in a subject including administering a pharmaceutically effective amount of a compound or composition that inhibits platelet aggregation and specifically, thrombin induced platelet aggregation.

The invention included a method for treating or preventing a disease or condition of platelet aggregation in a subject by administering a pharmaceutically effective amount of a compound represented by formula (I):

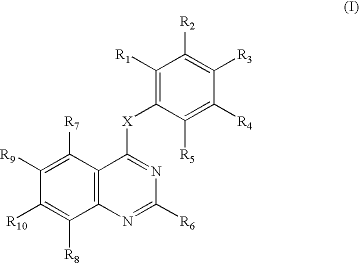

wherein:

X is selected from the group consisting of HN, R₁₁N, S, O, CH₂, and R₁₁CH;

R₁₁ is (C₁-C₄)alkyl or (C₁-C₄)alkanoyl;

R₁-R₅ are each independently selected from the group consisting of hydrogen, hydroxy, and halo where at least one of R₁-R₅ is hydroxy;

R₆, R₇ and R₈ are each independently selected from the group consisting of hydrogen, hydroxy, mercapto, amino, nitro, (C₁-C₄)alkyl, (C₁-C₄)alkoxy, (C₁-C₄)alkylthio, and halo; and

R₉ and R₁₀ are each independently selected from the group consisting of hydrogen, (C₁-C₄)alkyl, (C₁-C₄)alkoxy, halo, and (C₁-C₄)alkanoyl; or R₉ and R₁₀ together are methylenedioxy; or a pharmaceutically acceptable salt thereof.

More particularly, the invention includes a method for treating or preventing a disease or condition of platelet aggregation in a subject by administering a pharmaceutically effective amount of a compound represented by formula (II):

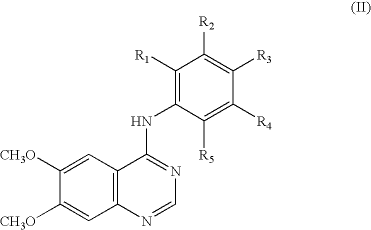

wherein:

R₁-R₅ are each independently selected from the group consisting of hydrogen, hydroxy, and halo where at least one of R₁-R₅ is hydroxy; and a pharmaceutically acceptable salt thereof.

Additional advantages of the invention will be set forth in part in the description which follows, and in part will be obvious from the description, or may be learned by practice of the invention as herein described. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The accompanying drawings, which are incorporated in and constitute a part of this specification, illustrate several experimental examples and together with the description, serve to explain the principles of the invention.

### BRIEF DESCRIPTION OF THE DRAWINGS

FIG. 1 is a computer image of a gel comparing PCR products derived from mice of the wild type JAK3^{+/+} homozygous genotype and homozygous knockout JAK3^{-/-}.

FIGS. 2A-2G are computer images of Western blots showing JAK3-dependent tyrosine phosphorylation of STAT1 and STAT3 in thrombin-stimulated platelets.

FIGS. 2A and 2B show results for whole cell lysates from control and JAK3-deficient mouse platelets stimulated with thrombin and probed with anti-STAT1 antibodies raised against phosphorylated STAT 1 (FIG. 2A, upper panel) and STAT 1 (FIG. 2A, lower panel) and phosphorylated STAT 3 (FIG. 2B, upper panel) and STAT 3 (FIG. 2B, lower panel).

FIG. 2C shows results of STAT 1 immunoprecipitated from human platelets stimulated with thrombin and probed with antibodies raised against phospho-tyrosine (FIG. 2C, upper panel) and STAT1 (FIG. 2C, lower panel).

FIG. 2D shows results of STAT3 immunoprecipitated from human platelets, stimulated with thrombin and probed with antibodies raised against phosphorylated STAT 3 (FIG. 2D, upper panel) and STAT 3 (FIG. 2D, lower panel).

FIG. 2E shows results of JAK 3 immunoprecipitated from platelets stimulated with thrombin after treatment with vehicle or WHI-P131. The immunoprecipitates were subjected to quantitative kinase assays (FIG. 2E, upper panel) and probed with an anti-JAK 3 antibody (FIG. 2E, lower panel).

FIGS. 2F and 2G show results of human platelets pretreated with vehicle or WHI-P131 prior to thrombin stimulation. FIG. 2F shows STAT 1 immunoprecipitated from platelets stimulated with thrombin and probed with antibodies raised against phospho-tyrosine (FIG. 2F, upper panel) and STAT1 (FIG. 2F, lower panel). FIG. 2G shows whole cell lysates from platelets stimulated with thrombin and probed with antibodies raised against phosphorylated STAT 3 (FIG. 2G, upper panel) and STAT 3 (FIG. 2G, lower panel).

FIGS. 3A and 3B are computer images of Western blots showing the effects of WHI-P131 on thrombin-induced translocation of TX-100 soluble proteins to the membrane-associated cytoskeleton. Human platelets were pretreated with vehicle (FIG. 3A) or WHI-P131 (FIG. 3B) for 30 minutes and then stimulated with thrombin. Treated platelets were fractionated into cytoplasmic and TX-100 soluble and TX-100 insoluble fractions and probed with antibodies raised against JAK3, tubulin, actin, STAT1, STAT 3 and SYK.

FIGS. 4A-4D are computer topographical images of platelet surface membranes showing the effects of WHI-P131 on thrombin-induced shape change in platelets. FIG. 4A shows resting platelets with a discoid appearance and smooth contours; FIG. 4B shows vehicle-pretreated control platelets stimulated with thrombin; FIG. 4C shows WHI-P131 pretreated, unstimulated platelets; FIG. 4D shows WHI-P 131 pretreated platelets stimulated with thrombin.

FIGS. 5A-5D are computer images of transmission electron micrographs (TEM) showing the effects of WHI-P131 on thrombin-induced ultrastructural changes and degranulation in platelets. FIG. 5A shows untreated, unstimulated control platelets with a typical discoid appearance and disperse distribution of granules; FIG. 5B shows vehicle-treated, thrombin-stimulated platelets with spike-like pseudopodia and coalescence of granules in the center; FIG. 5C shows WHI-P131-treated unstimulated platelets; FIG. 5D shows WHI-P131-treated, thrombin-stimulated platelets with the largely discoid appearance of resting platelets; FIG. 5E is a graph showing serotonin release from thrombin-stimulated platelets.

FIG. 6 is a graph showing the role of jak3 in thrombin-induced platelet aggregation. Representative aggregation curves of platelets from JAK3-knockout mice and C57BL/6 wild type mice are shown for thrombin induced platelet aggregation in citrated whole blood measured by optical impedence.

FIGS. 7A-7D are graphs showing the effects of the JAK3 inhibitor WHI-P131 on thrombin-induced platelet aggregation. FIG. 7A shows a composite concentration-inhibitory effect curve for WHI-P131. Results are expressed as the percent control of thrombin-induced maximum platelet aggregation as a function of the applied WHI-P131 concentration. Shown are representative traces of aggregation curves of platelets treated with WHI-P131 FIG. 7B) or WHI-P258 (FIG. 7C) or vehicle (Control) and then stimulated with thrombin (0.1 U/mL). FIG. 7D demonstrates that WHI-P131 does not inhibit collagen-induced platelet aggregation.

FIG. 8 is a graph showing the protective effects of WHI-P131 in a mouse model of fatal thromboembolism. Shown are the cumulative proportions of mice surviving event-free 3 minutes, 6 minutes and 48 hours after the injection of thromboplastin. Error bars represent the SEM values. * p<0.05, Log-rank test.

### DESCRIPTION OF THE PREFERRED EMBODIMENTS

The present invention may be understood more readily by reference to the following detailed description of embodiments and preferred embodiments of the invention, and the Examples included therein and to the Figures and their previous and following description.

In this specification and in the claims that follow, reference will be made to a number of terms that shall be defined to have the following meanings:

Reference in the specification and concluding claims to parts by weight of a particular component in a composition, denotes the weight relationship between the component and any other components in the composition for which a part by weight is expressed.

The term “halogen” or “halo” refers to bromine, chlorine, fluorine, and iodine.

The term “alkyl” as used herein refers to a branched or unbranched saturated hydrocarbon group, such as methyl, ethyl, n-propyl, isopropyl, n-butyl, isobutyl, t-butyl, octyl, decyl, tetradecyl, hexadecyl, eicosyl, tetracosyl and the like. The alkyl group may have one or more hydrogen atoms replaced with a functional group. The term “cycloalkane” as used herein refers to a cyclic alkane group.

The term “alkoxy” as used herein intends an alkyl group bound through a single, terminal ether linkage; that is, an “alkoxy” group may be defined as-OR where R is alkyl as defined above. An “alkylthio” group intends an alkyl group bound through a sulfur linkage such as—SR where R is alkyl as defined above.

The term “alkanoyl” as used herein refers to a branched or unbranched acyl group, a carbonyl group with an alkyl group attached. The general formula for alkanoyl is R—CO— wherein the carbon atom is linked to the compound. Example alkanoyls include methanoyl (formyl), ethanoyl (acetyl), propanoyl, and benzoyl.

The term “mercapto” as used herein refers to an —SH group. “Amino” refers to a —NH₂ group, and nitro refers to a NO₂ group.

As used herein, the term “STAT-3” means signal transducers and activators of transcription (STAT) that associate with JAK-3, including STAT-3α (p92) and STAT-3β (p83) isoforms.

By “platelet aggregation” is meant the clumping together of platelets or red blood cells. As used herein, “inhibiting platelet aggregation” includes slowing platelet aggregation, as well as completely eliminating and/or preventing platelet aggregation. Additionally, “inhibiting platelet function” includes decreasing platelet function, as well as completely eliminating and/or preventing the platelet function.

Conditions of platelet aggregation include, but are not limited to, embolus formation, thrombolytic complications, disseminated intravascular comgelopathy, thrombosis, coronary heart disease, thromboembolic complications, myocardial infarction, restenosis, and atrial thrombosis formation in atrial fibrillation, chronic unstable angina, transient ischemic attacks and strokes, peripheral vascular disease, arterial thrombosis, preeclampsia, embolism, restenosis and/or thrombosis following angioplasty, carotid endarterectomy, anastomosis of vascular grafts, and chronic exposure to cardiovascular devices. Such conditions may also result from thromboembolism and reocculsion during and after thermbolytid therapy, after angioplasty, and after coronary artery bypass.

“Thrombin induced platelet aggregation” includes platelet aggregation in response to the enzyme thrombin, which is formed in blood from prothrombin.

“Collagen induced platelet aggregation” includes platelet aggregation in response to the protein collagen.

As used throughout, “contacting” is meant an instance of exposure of at least one cell (e.g., a neural cell, a stem cell, a cardiac cell) to an agent (e.g., a compound that inhibits platelet aggregation and specifically, thrombin induced platelet aggregation).

The term “subject” is meant an individual. Preferably, the subject is a mammal such as a primate, and more preferably, a human. Thus, the “subject” can include domesticated animals (e.g., cats, dogs, etc.), livestock (e.g., cattle, horses, pigs, sheep, goats, etc.), and laboratory animals (e.g., mouse, rabbit, rat, guinea pig, etc.).

In general, “pharmaceutically effective amount” or “pharmaceutically effective dose” means the amount needed to achieve the desired result or results (treating or preventing platelet aggregation). One of ordinary skill in the art will recognize that the potency and, therefore, a “pharmaceutically effective amount” can vary for the various compounds that inhibit platelet aggregation and specifically, thrombin induced platelet aggregation used in the invention. One skilled in the art can readily assess the potency of the compounds.

By “pharmaceutically acceptable” is meant a material that is not biologically or otherwise undesirable, i.e., the material may be administered to an individual along with the selected compounds without causing any undesirable biological effects or interacting in a deleterious manner with any of the other components of the pharmaceutical composition in which it is contained.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine; diethylamine, triethylamine, tripropylamine, ethanolamine, 2-dimethylaminoethanol, 2-diethylaminoethanol, lysine, arginine, histidine, and the like. The reaction is conducted in water, alone or in combination with an inert, water-miscible organic solvent, at a temperature of from about 0° C. to 100° C., preferably at room temperature. The molar ratio of the compound that inhibits platelet aggregation and specifically, thrombin induced platelet aggregation, to base used are chosen to provide the ratio desired for any particular salts. For preparing, for example, the ammonium salts of the free acid starting material, a particular preferred embodiment, the starting material can be treated with approximately one equivalent of base to yield a salt. When calcium salts are prepared, approximately one half a molar equivalent of base is used to yield a neutral salt, while for aluminum slats, approximately one-third a molar equivalent of base will be used.

Ester derivatives are typically prepared as precursors to the acid form of the compounds, and accordingly may serve as prodrugs. Generally, these derivatives will be alkyl esters such as methyl, ethyl, and the like. Amide derivatives —(CO)NH₂, —(CO)NHR and —(CO)NR₂, where R is alkyl, may be prepared by reaction of the carboxylic acid-containing compound with ammonia or a substituted amine.

Specific values listed below for radicals, substituents, and ranges, are for illustration only; they do not exclude other defined values or other values within defined ranges for the radicals and substituents.

Preferred constituents of R₁-R₅ for the compounds of formula I are independently hydrogen, hydroxy, and halo with at least one of R₁-R₅ being hydroxy; and preferred constituents of R₆, R₇, and R are each independently selected from the group consisting of hydrogen, hydroxy, mercapto, amino, nitro, (C₁-C₄)alkyl, (C₁-C₄)alkoxy, (C₁-C₄)alkylthio, and halo; more preferably, R₆, R₇, and R₈ are each hydrogen.

Preferably, R₉ and R₁₀ are each independently selected from the group consisting of hydrogen, (C₁-C₄)alkyl, (C₁-C₄)alkoxy, halo, and (C₁-C₄)alkanoyl; or R₉ and R₁₀ together are methylenedioxy; more preferably R₉ and R₁₀ are each OCH₃.

Preferred constituents of X are BN, R₁₁N, S, O, CH₃ and R₁₁CH; wherein R₁₁ is preferably (C₁-C₄)alkyl or (C₁-C₄)alkanoyl; more preferably X is HN.

Some exemplary compounds of the invention are listed below with their characterization data:

4-(3′,5′-dibromo4′-hydroxyphenyl)-amino-6,7-dimethoxyquinazoline[WHI-P97]

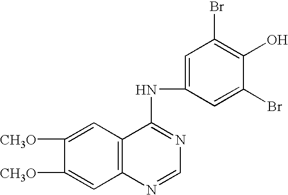

m.p.>300.0° C. UV(MeOH)λₘₐₓ: 208.0, 210.0, 245.0 , 320.0 nm; IR(KBr)υₘₐₓ: 3504(br), 3419, 2868, 1627, 1512, 1425, 1250, 1155 cm⁻¹; ¹H NMR(DMSO-d₆): δ9.71(s, 1H, —NH), 9.39(s, 1H, —OH), 8.48(s, 1H, 2-H), 8.07(s, 2H, 2′, 6′-H), 7.76(s, 1H, 5-H), 7.17(s, 1H, 8-H), 3.94(s, 3H, —OCH₃), 3.91(s, 3H, —OCH₃). GC/MS m/z 456(M⁺+1,54.40), 455(M⁺, 100.00), 454(M⁺−1,78.01), 439(M⁺ —OH, 7.96), 376(M⁺+1 —Br, 9.76), 375(M⁺ —Br, 10.91), 360(5.23). Anal. (C₁₆H₁₃Br₂N₃O₃) C, H, N.

4-(4′-Hydroxyphenyl)-amino-6,7-dimethoxyquinazoline[WHI-P131]

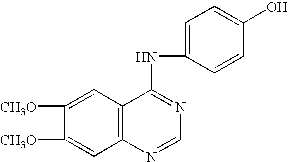

m.p. 245.0-248.0.° C. UV(MeOH)λₘₐₓ: 203.0, 222.0, 251.0, 320.0 nm; IR(KBr)υₘₐₓ: 3428, 2836, 1635, 1516, 1443, 1234 cm⁻¹; ¹H NMR(DMSO-d₆): δ11.21(s, 1H, —NH), 9.70(s, 1H, —OH), 8.74(s, 1H, 2-H), 8.22(s, 1H, 5-H), 7.40(d, 2H, J=8.9 Hz, 2′,6′-H), 7.29(s, 1H, 8-H), 6.85(d, 2H, J=8.9 Hz, 3′,5′-H), 3.98(s, 3H, —OCH₃), 3.97(s, 3H, —OCH₃). GC/MS m/z 298 (M⁺+1, 100.00), 297(M⁺, 26.56), 296(M⁺−1, 12.46). Anal. (C₁₆H₁₅N₃O₃HCl) C, H, N.

4-3′-Bromo-4′-hydroxyphenyl)-amino6,7-dimethoxyquinazoline[WH-P154]

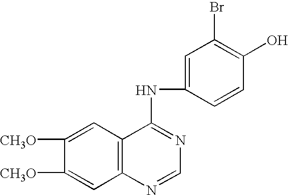

m.p. 233.0-233.5° C. UV(MeOH)λₘₐₓ: 203.0, 222.0, 250.0, 335.0 nm; IR(KBr)υₘₐₓ: 3431 br, 2841,1624, 1498, 1423, 1244 cm⁻¹; ¹H NMR(DMSO-d₆): δ10.08(s, 1H, —NH), 9.38(s, 1H, —OH), 8.40(s, 1H, 2-H ), 7.89(d, 1H,J_{2′,5′}=2.7 Hz, 2′-H), 7.75(s, 1H, 5-H), 7.55(dd, 1H, J_{5′,6′}=9.0 Hz, J_{2′,6′}=2.7 Hz,, 6′-H), 7.14(s, 1H, 8-H), 6.97(d, 1H,J_{5′,6′}=9.0 Hz, 5′-H), 3.92(s,3H, —OCH₃), 3.90(s, 3H, —OCH₃). GC/MS rn/z 378(M⁺+2, 90.68), 377(M⁺+1, 37.49), 376(M⁺, 100.00), 360(M⁺, 3.63), 298(18.86), 282 (6.65).

Anal. (C₁₆H₁₄N₃O₃HCl) C, H, N.

4-(3′-Hydroxyphenyl)-amino-6,7-dimethoxyquinazoline[WHI-P1801]

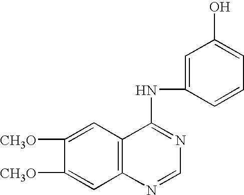

m.p. 256.0-258.0° C. ¹HNMR(DMSO-d₆): δ9.41(s, 1H, —NH), 9.36(s, 1H, —OH), 8.46(s, 1H, 2-H), 7.84(s, 1H, 5-H), 7.84−6.50(m, 4H, 2′, 4′, 5′, 6′-H), 7.20(s, 1H, 8-H), 3.96(s, 3H, —OCH₃), 3.93(s, 3H, —OCH₃). UV(MeOH)λₘₐₓ(ε): 204.0, 224.0, 252.0, 335.0 nm. IR(KBr)υₘₐₓ: 3394, 2836, 1626, 1508, 1429, 1251 cm¹. GM/MS m/z: 297(M⁺, 61.89), 296(M⁺, 61.89), 296(M⁺−1, 100.00), 280(M⁺ —OH, 13.63). Anal. (C₁₆H₁₅N₃O₃. HCl) C, H, N.

A preferred compound for use in the present invention is 4(3′-bromo-4′-hydroxyphenyl)amino-6,7-dimethoxyquinazoline,

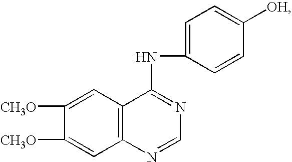

or a pharmaceutically acceptable salt thereof.

Pharmaceutically acceptable salts of 4(4′-hydroxyphenyl)amino-6,7-dimethoxyquinazoline, or any other compound useful in the present invention, may be used in the present invention. Examples of acceptable salts are organic acid addition salts formed with acids, which form a physiological acceptable anion, including, but not limited to, tosylate, methanesulfonate, acetate, citrate, malonate, tartarate, succinate, benzoate, ascorbate, α-ketoglutarate, and α-glycerophosphate. Suitable inorganic salts may also be formed, including, but not limited to, hydrochloride, sulfate, nitrate, bicarbonate, and carbonate salts.

Acceptable salts may be obtained using standard procedures well known in the art, for example by reacting a sufficiently basic compounds such as an amine with a suitable acid affording a physiologically acceptable anion.

Synthetic Methods:

The compounds of the present invention may be readily synthesized using techniques generally known to synthetic organic chemists. Suitable experimental methods for making and derivatizing aromatic compounds are described, for example, in U.S. Pat. No. 6,080,748 to Uckun et al., the disclosure of which is hereby incorporated by reference.

Utility and Administration:

The therapeutic method included herewith is useful for treating or preventing a condition of platelet aggregation, in a subject comprising administering a pharmaceutically effective amount of a compound or composition that inhibits JAK-3 and/or tyrosine phosphorylation of STAT-3 and that inhibits platelet aggregation, specifically, thrombin induced platelet aggregation.

The condition of platelet aggregation includes hematopoietic and cerbrovascular diseases such as, but not limited to, embolus formation, thrombolytic complications, disseminated intravascular comgelopathy, thrombosis, coronary heart disease, thromboembolic complications, myocardial infarction, restenosis, or atrial thrombosis formation in atrial fibrillation. Such platelet aggregation inhibition may selectively target the thrombin pathway, over other pathways including collagen induced platelet aggregation.

The methods include contacting the cells with such compounds or compositions, or administering to the subject a pharmaceutically effective amount of these compounds or compositions. In one embodiment, the cells are part of the blood and immune system including: red blood cell, megakaryocytes, macrophages (e.g. monocytes, connective tissue macrophages, Langerhans cells, osteoclasts, dendritic cells, microglial cells), neutrophils, eosinophils, basophils, mast cells, T lymphocytes (e.g. helper T cells, suppressor T cells, killer T cells), B lymphocytes (e.g. IgM, IgG, IgA, IgE), killer cell, and stem cells and committed progenitors for the blood and immune system. In another embodiment, the cells are contractile cells such as skeletal muscle cells (e.g. red, white, intermediate, muscle spindle, satellite cells), heart muscle cells (e.g. ordinary, nodal, Purkinje fiber), smooth muscle cells, and myoepithelial cells.

It is well known in the art how to determine the inhibition of platelet aggregation using the standard tests described herein, or using other similar tests. Preferably, the method would result in at least a 10% reduction in thrombin-induced platelet aggregation, including, for example, 15%, 20%, 25%, 30%, 40%, 50%, 60%, 70%, 80%, 90%, 100%, or any amount in between, more preferably by 90%. Similarly, the method would result in at least a 10% reduction in thrombin-induced tyrosine phosphorylation of STAT-3β, including, for example, 15%, 20%, 25%, 30%, 40%, 50%, 60%, 70%, 80%, 90%, 100%.

The reduction can be measured, for example, by comparing the optical impedence in a chronology platelet aggregometer. Any other known measurement method may also be used. For example, upon thrombin stimulation, STAT-3β tyrosine phosphorylation increases over time and so the measurement may include measuring JAK-3 and/or STAT-3β tyrosine phosphorylation.

The cells can be contacted ill vitro, for example, by adding the compound to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). The duration of “contact with a cell or population of cells is determined by the time the compound is present at physiologically effective levels or at presumed physiologically effective levels in the medium or extracellular fluid bathing the cell or cells. Preferably, the duraton of contact is 1-96 hours, and more preferably, for 24 hours, but such time would vary based on the half life of the compound and could be optimized by one skilled in the art using routine experimentation.

The compounds useful in the present invention can be formulated as pharmaceutical compositions and administered to a mammalian host, such as a human patient or a domestic animal in a variety of forms adapted to the chosen route of administration, i.e., orally or parenterally, by intravenous, intramuscular, topical or subcutaneous routes.

The compounds of the present invention can also be administered using gene therapy methods of delivery. See, e.g., U.S. Pat. No. 5,399,346, which is incorporated by reference in its entirety. Using a gene therapy method of delivery, primary cells transfected with the gene for the compounds of the present invention can additionally be transfected with tissue specific promoters to target specific organs, tissue, grafts, tumors, or cells.

Thus, the present compounds may be systemically administered, e.g., orally, in combination with a pharmaceutically acceptable vehicle such as an inert diluent or an assimilable edible carrier. They may be enclosed in bard or soft shell gelatin capsules, may be compressed into tablets, or may be incorporated directly with the food of the patient's diet. For oral therapeutic administration, the active compounds may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compounds may be incorporated into sustained-release preparations and devices.

The active compounds may also be administered intranasally by inhalation, intravenously or intraperitoneally by infusion or injection. Solutions of the active compounds or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for inhalation, injection, or infusion can include sterile aqueous solutions or dispersions or sterile powders comprising the active ingredient which are adapted for the extemporaneous preparation of sterile inhalation, injectable, or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohollglycol blends, in which the present compounds can be dissolved or dispersed at effective levels, optionally with the aid of non-toxic surfactants. Adjuvants such as fragrances and additional antimicrobial agents can be added to optimize the properties for-a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortztman (U.S. Pat. No. 4,820,508).

Useful dosages of the compounds can be determined by comparing their in vitro activity, and ill vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compounds, or an active salt or derivative thereof, required for use in treatment will vary not only with the particular salt selected but also with the route of administration, the nature of the condition being treated and the age and condition of the patient and will be ultimately at the discretion of the attendant physician or clinician. Also the dosage of the compound varies depending on the target cell, tumor, tissue, graft, or organ.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound may conveniently be administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.0005 to about 300 μM, preferably, about 0.001 to 100 μM, more preferably, about 1 to about 100 μM. This may be achieved, for example, by the intravenous injection of a concentration of the active ingredient, optionally in saline, or orally administered as a bolus. Desirable blood levels may be maintained by continuous infusion to provide about 0.0005-50.0 mg/kg/hr or by intermittent infusions containing about 0.004-150 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

### Example 1

JAK3 Dependent Tyrosine Phosphorylation of STAT 1 and STAT3 Proteins in Thrombin-Stimulated Platelets

The effects of thrombin stimulation on the phosphorylation status of STAT1 and STAT3 proteins in platelets was determined using platelets from wild-type C57BL/6 mice and from JAK3 deficient platelets from JAK3-knockout mice using the procedures described below.

Mice

Control C57BL/6 mice were purchased from Taconic (Germantown, N.Y.). A breeder pair of JAK3-knockout mice (JAK3^{-/-}, C57BL/6×129/Sv, H-2^{b}) (11), A011 (male) and A1038 (female) were obtained from Dr. J. N. Ihle, St. Jude Children's Research Hospital, Memphis, Tenn. These mice were created by the targeted disruption of the JAK3 gene through homologous recombination using the hygromycin-resistance gene (Hyg) cassette(Nosaka, et.al., 1995 Science270(5237), 800-2). These founder JAK3^{-/-} mice were bred to C57BL/6 mice (Jackson Laboratory, Bar Harbor, Me.) and the offspring of the F1 generation were back-crossed to C57BL/6 mice. After three generations of back-crossing to C57BL/6 mice, the offspring were inter-crossed to produce homozygote JAK3^{-/-} and wild-type (WT) JAK3 ^{+/+} mice.

The genotype of mice was confirmed by multiplex polymerase chain reaction (PCR) tests. In brief, a 0.5 inch (1.27 cm) tail tissue section was taken from each mouse and digested at 55° C. in 600 μL lysis buffer (50 mM Tris pH 8.0, 100 mM EDTA, 100 mM NaCl, 1% SDS) with 50 μL Proteinase K (10 mg/mL). Genomic DNA was purified with phenol and chloroform extractions and ethanol precipitation(12).

Three primers were employed in the PCR tests: a 30-base primer JAK3-S (5′-ACC TAG TCC CCA GCT TGG CTG TCA CTT GGG-3′)[SEQ ID NO: 1], a 30-base primer JAK3-AS (5′-CAA AGC GGT GAC ATG TCT CCA GCC CAA ACC-3′) [SEQ ID NO: 2], and a 30-base primer JAK3-Hyg (5′-ATG GTT TTT GGA TGG CCT GGG CAT GGA CCG-3′)[SEQ ID NO: 3] (Biosynthesis, Lewisville, Tex.-100).

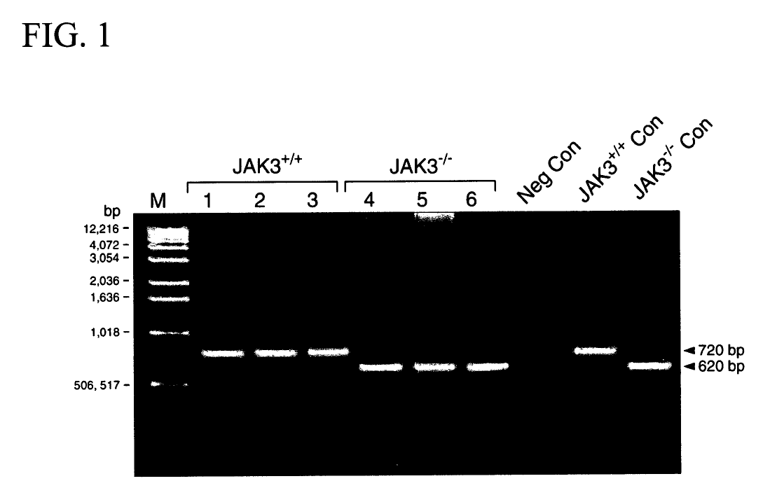

The JAK3-AS×JAK3-Hyg PCR primer pair yielded a 620 bp “mutant” PCR product in tissues from JAK3^{-/-} mice. The JAK3-AS×JAK3-S PCR primer pair yielded a 720 bp “wild type” PCR product in tissues from homozygote JAK3^{+/+} or heterozygote JAK3^{+/-} mice. Homozygous JAK3^{+/+} genotype was documented by a single 720 bp PCR product and homozygous JAK3^{-/-} genotype was documented by a single 620 bp PCR product. Heterozygous JAK3^{+/-} genotype was documented by the presence of both 720 bp and 620 bp PCR products (see FIG. 1).

Each 50 μL PCR reaction medium consisted of 1×PCR buffer II containing 2.5 mM MgCl₂ (Perkin Elmer's Amplitaq Gold Kit), 0.2 mM dNTP, (Boehringer Mannheim), 0.4 μM of each primer, 6% DMSO, and 2.5 U AmpliTaq Gold enzyme. The PCR conditions were 94° C. for 10 minutes, 30 cycles [94° C. for 1 minute, 57° C. for 1 minute, 72° C. for 1 minute with a 5 second extension], then 72° C. for 10 minutes (Touchdown, Hybaid, 11044 Rutledge Drive, N. Potomac, Md.). The PCR products were cloned into the original TA cloning vector (Invitrogen, Karlsbad, Calif.). Sequence analysis was accomplished by thermosequenase PCR (Amersham Pharmacia Biotech, Piscataway, N.J.) using Cy-5 labeled T3 and T7 sequencing primers (IDT, Coralville, Iowa). DNA sequences were analyzed against published JAK3 DNA sequence using Lasergene software (DNAStar, Madison, Wis.).

Immunoprecipitation and Western Blotting Analysis

Platelets were isolated from PRP (Memorial Blood Bank, Minneapolis, Minn.) as previously described (Asselin, et.al., 1997 Blood 89(4) 123542) and resuspended at a concentration of 3×10⁹ cells/mL in a modified Tyrode's buffer (137 mM NaCl, 2.7 mM KCl, 0.9 mM MgCl₂, 5.5 mM glucose, 3.3 mM NaH₂PO₄, 3.8 mM Hepes, pH 7.4). Platelets were incubated with indicated concentrations of WHI-P131 or vehicle (PBS supplemented with 1% DMSO) for 30 minutes at 37° C. Platelets were then stimulated at 37° C. with 2 μg/mL (or 10 μg/mL) collagen or 0.1 U/mL thrombin (Chronolog Inc., Philadelphia, Pa.). Stimulation was stopped and platelets were lysed at the indicated time points by adding ice cold 3× Triton X-100 lysis buffer (150 mM NaCl, 15 mM EGTA, 3% Triton X-100, 3% Sodium deoxycholate, 0.3% SDS, 3 mM PMSF, 3 mM Na₃VO₄, 60 μg/mL leupeptin, 60 μg/mL aprotinin, 50 mM Tris-HCl pH 7.4) and incubating for 1 hour on ice.

Following removal of the membranous fraction by centrifgation (12,000×g, 30 min) the samples were subjected to immunoprecipitation utilizing antibodies raised against JAK 3 and STAT1 (Santa Cruz, Santa Cruz, Calif.), or STAT3 (Transduction Labs, Lexington, Ky.) (Vassilev, et.al., 1999, J Biol Chem 274(3), 1646-56). Immunoprecipitations, immune-complex protein kinase assays, and immunoblotting on PVDF membranes (Milipore, Bedford, Mass.) using the ECL chemiluminescence detection system (Amersham Life Sciences, Arlington Heights, Ill.) were conducted as described previously (Goodman, et.al., 1998 J Biol Chem 273(28), 17742-8). For immunoblotting, antibodies against phosphotyrosine and JAK3, STAT1, STAT3, phospho-STAT1 and phospho-STAT3 were used as obtained from New England BioLabs, Beverly, Mass. Horseradish peroxidase-conjugated sheep antimouse, donkey anti-rabbit secondary antibodies were purchased from Transduction Laboratories (Lexington, Ky.). Horseradish peroxidase-conjugated sheep anti-goat antibodies were purchased from Santa Cruz (Santa Cruz, Calif.).

Following electrophoresis, kinase gels were dried onto Whatman 3M filter paper and subjected to phosphorimaging on a Molecular Imager (Bio-Rad, Hercules, Calif.) as well as autoradiography on film. Similarly, all chemiluminescent JAK3 Western blots were subjected to three dimensional densitometric scanning using the Molecular Imager and Imaging Densitometer using the Molecular Analyst/Macintosh version 2.1 software following the specifications of the manufacturer (Bio-Rad). A JAK 3 kinase activity index was determined by comparing the ratios of the kinase activity in phosphorimager units (PIU) and density of the protein bands in densitometric scanning units (DSU) to those of the baseline sample using the formula: Activity Index=[PIU of kinase band/DSU of JAK3 protein band]_{test sample}. Stimulation index=[PIU of kinase band/DSU of JAK 3 protein band]_{test sample}: [PIU of kinase band/DSU of JAK3 protein band]_{baseline control sample}.

Results

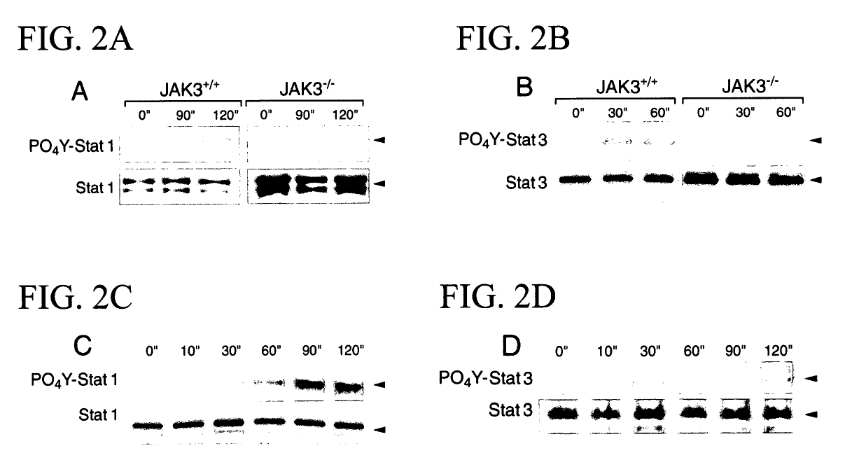

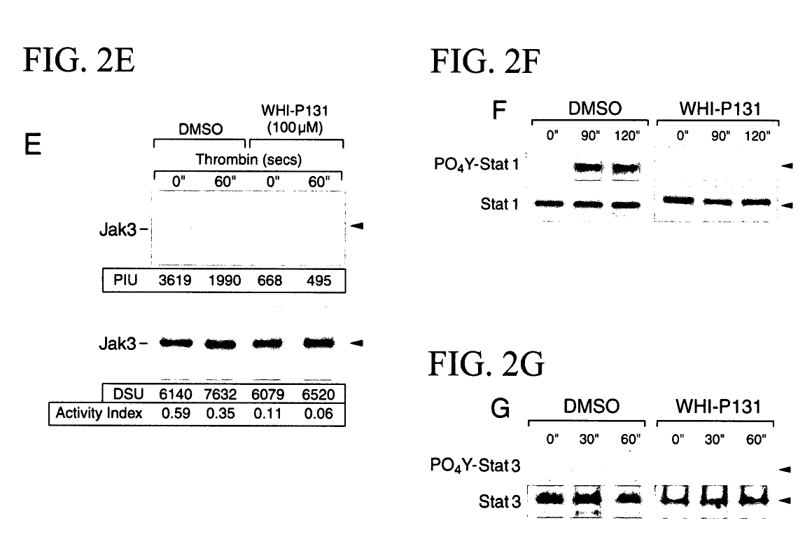

As show in in FIGS. 2A-2B, treatment of platelets with 0.1 U/mL thrombin resulted in induced tyrosine phosphorylation of both STAT1 (FIG. 2A) and STAT3 (FIG. 2B) proteins. Thrombin-induced tyrosine phosphorylation of STAT1 and STAT3 were JAK3 dependent, because thrombin stimulation failed to induce tyrosine phosphorylation of these STAT proteins in JAK3-deficient platelets from JAK3-knockout (JAK3^{-/-}) mice. Similarly, stimulation of human platelets with 0.1 U/mL thrombin enhanced the tyrosine phosphorylation of STAT1 and STAT3 proteins (FIGS. 2C-2D).

Pretreatment of human platelets with the JAK3 inhbitory WHI-P131 (100 micromolar) markedly decreased the baseline enzymatic activity of constitutively active JAK3, as measured by autophosphorylation (FIG. 2E), and abolished the thrombin-induced tyrosine phosphorylation of STAT1 and STAT3 (FIGS. 2F-2G).

### Example 2

JAK3 Inhibitor Inhibits Thrombin-Induced Platelet Aggregation

Platelet Aggregation Assays

Platelet-rich plasma (PRP) was purchased from the Memorial Blood Bank (Minneapolis, Minn.) and used according to the guidelines of the Parker Hughes Institute Human Subjects Committee. The PRP samples were treated with varying concentrations of WHI-P131 for 20 minutes at 37° C. Control PRP samples were treated with vehicle alone. The treated PRP samples were diluted 1:4 with sterile normal saline and platelets were stimulated with thrombin (0.1 U/mL, Chronolog Inc., Philadelphia, Pa.) under stirred conditions. Platelet aggregation was monitored in a platelet aggregometer (Model 560 Dual Chamber Instrument, Chronolog Inc., Philadelphia, Pa.) for 5 minutes. The IC₅₀ values for WHI-P131-mediated inhibition of agonist-induced platelet aggregation were calculated by non-linear regression analysis using Graphpad Prism software version 2.0 (Graphpad Software, Inc., San Diego, Calif.).

For optical impedence aggregation studies, blood was extracted from JAK 3-knockout and control C57BL/6 mice by eye bleeds into tubes containing 15% v/v ACD (0.8% w/v citric acid, 2.2% w/v trisodium citrate, 2.45 % w/v dextrose) and mixed gently to prevent coagulation. Citrated blood was diluted with an equal volume of saline and prewarmed at 37° C. for 5 minutes. The platelet agonist, thrombin (0.1 U/mL) was added at 1 minute to induce aggregation. Thrombin-induced platelet aggregation was measured from wild type and knockout mice (n=3 for each type) in a Whole Blood Platelet Aggregometer (Model 560 Dual Chamber, Chronolog Inc., Philadelphia, Pa.).

Cytoskeletal Fractionation

Platelets (1×10⁸/mL) were treated with inhibitor (100 μM, 30 minutes, 37° C.) or vehicle (1% DMSO) and stimulated with thrombin (0.1 U/mL) or collagen (10 μg/mL). Isolation of the cytoplasmic and TX-100 soluble and insoluble fractions was performed as previously described (Hirao, et.al., 1997 Embo J. 16(9), 2342-51; Oda, et.al., 1992 J Biol Chein 267(28), 20075-81). Fractions were analyzed by Western blot analysis utilizing antibodies raised against JAK 3, STAT1, SYK (Santa Cruz, Santa Cruz, Calif.), STAT3 (Transduction Laboratories, Lexington, Ky.), tubulin and actin (Sigma, St. Louis, Mo.).

High-Resolution Low-Voltage Scanning Electron Microscopy (HR-LVSEM)

HR-LVSEM was utilized for topographical imaging of the platelet surface membrane, as previously reported ( D'Cruz, et.al., 1998 Biol Reprod 59(3), 503-15) . Aliquots of human platelets were incubated with 100 μM WHI-P131 or vehicle alone for 30 minutes. Treated platelets were then stimulated with thrombin (0.1 U/mL) for 10 seconds. Glutaraldehyde (3%) was added to stop the reaction. Samples were prepared for HR-LVSEM and analyzed using a Hitachi S-900 SEM instrument (Hitachi Instruments, Gaithersburg, Md.) at an accelerating voltage of 2 kV.

Transmission Electron Microscopy (TEM)

Aliquots of human platelets were incubated with 100 μM WHI-P131 or vehicle alone for 30 minutes and then stimulated with thrombin (0.1 U/mL) for 10 seconds. Samples were then prepared for TEM as previously described (White, J. (1983) in Methods in Hemotology (Harker L A, Z. T., ed), pp. 1-25, Churchhill Livingston, N.Y.). Briefly, 0.1% glutaraldehyde was added to stop the reaction. Following a brief centrifugation, the sample pellets were layered with 3% glutaraldehyde for 40 minutes at room temperature. The samples were then postfixed in 1% OsO₄ for 1 hour at 4° C., rinsed three times in distilled water at room temperature, dehydrated in a graded ethanol series (25, 50, 75, 90, 95 and 100%) and 100% propylene oxide. The samples were embedded in Embed 812 (Electron Microscopy Science, Washington, Pa.). Silver sections were picked up on mesh grids, stained 10 minutes in 1% uranyle acetate/70 % ethanol, and 10 minutes in Reynold's lead citrate. Sections were viewed in a JEOL 100× electron microscope at 60 kV. True magnifications were determined by photographing a calibration grid at each magnification step on the microscope and using this scale to determine final print enlargements.

Results

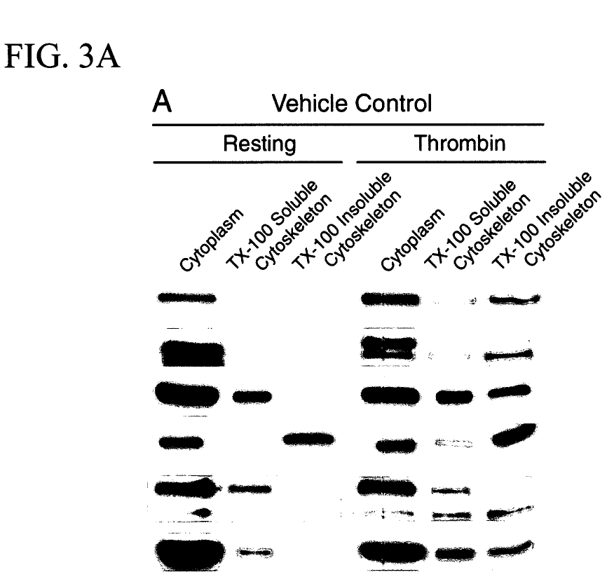

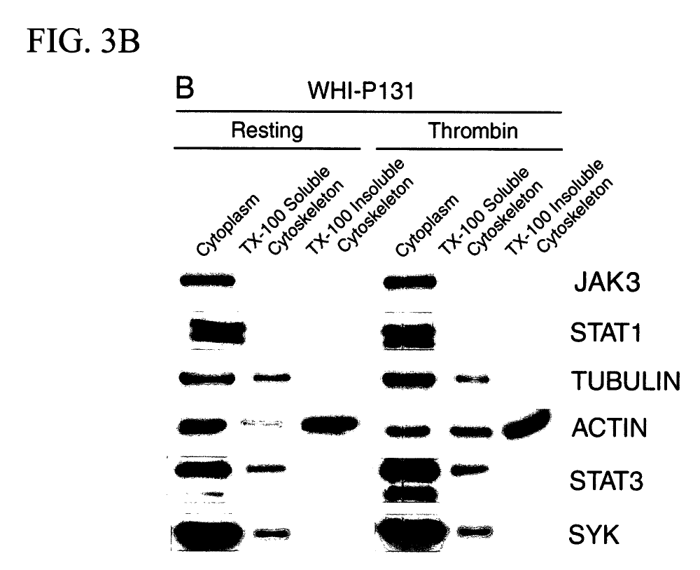

Activation of platelets after exposure to thrombin is associated with actin polymerization and rapid translocation of the tyrosine kinase SYK(Sada, et.al., 1997 Eur J Biochem 248(3), 827-33; Tohyama, et.al., 1994 Journal of Biological Chemistry 269(52), 32796-9) as well as tubulin to the TX-100 insoluble fraction that is associated with the actin filament network As shown in FIG. 3A, Western blot analysis of the cytoplasmic and TX-100 soluble and TX-100 insoluble fractions from unstimulated platelets confirmed the presence of abundant amounts of actin in the TX-100 insoluble fraction and SYK as well as tubulin in the TX-100 soluble (but not insoluble) fraction. Within 60 seconds after thrombin stimulation, a significant amount of SYK and tubulin translocated to the membrane associated cytoskeleton, as evidenced by the Western blot detection of SYK and tubulin in the actin-containing TX-100 insoluble fractions. Notably, thrombin stimulation also induced the translocation of JAK3, STAT1, and STAT3 proteins to the TX-100 insoluble fraction. Pretreatment of platelets with the JAK3 inhibitor WHI-P131 prevented the thrombin-induced relocalization of SYK, tubulin, JAK3, STAT1, as well as STAT3 to the TX-100 insoluble fractions (FIG. 3B).

The JAK-3 immune complexes immunoprecipitated from Triton-100 lysates of platelets treated with 100 μM WHI-P131 or DMSO and then stimulated with 0.1 U/ml thrombin were subjected to immune kinase assays. Additional JAK-3 immune complexes were collected and boiled in 2×SDS reducing sample buffer, fractionated on 8% polyacrylamide gels, transferred to PVDF membranes, and examined for the presence of JAK-3. The activity index was calculated by comparing the phosphoimager units (PIU) to the density of the protein bands in densitometric scanning units (DS) as shown in Table 1. The results indicate that JAK-3 kinase activity is significantly reduced by WHI-P131 treatment.

TABLE 1

| | DMSO | DMSO | WHI-P131 | WHI-P131 |
|---|---|---|---|---|
| Measure | 0 secs | 60 secs | 0 secs | 60 secs |
| PIU | 3619 | 1990 | 668 | 495 |
| DSU | 6140 | 7632 | 6079 | 6520 |
| Activity | 0.59 | 0.35 | 0.11 | 0.06 |

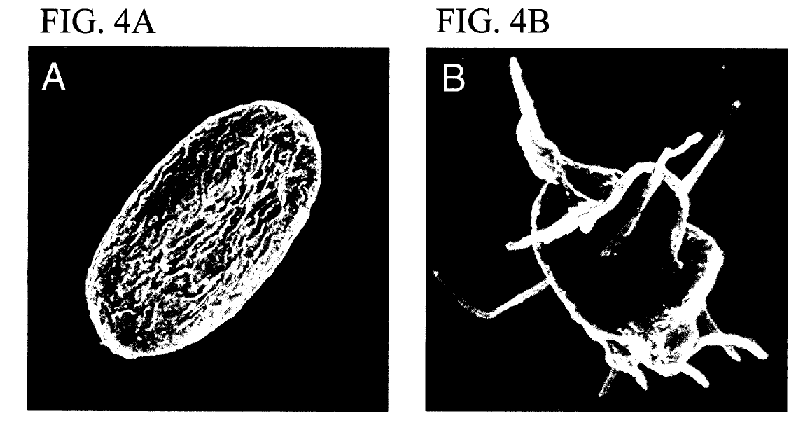

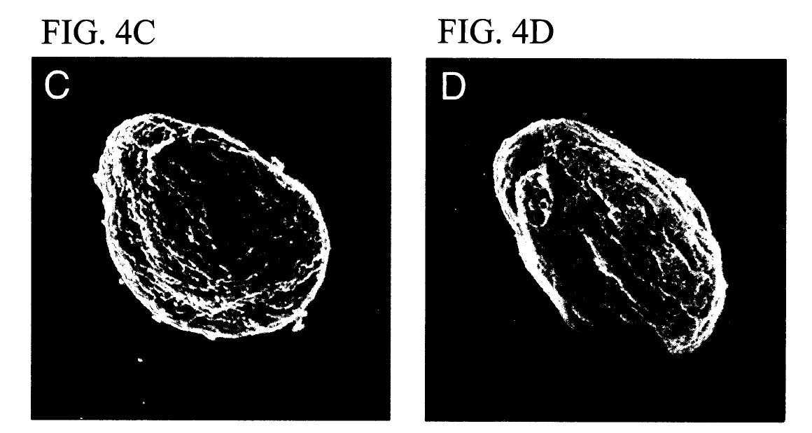

Platelet activation after thrombin stimulation was accompanied by marked changes in platelet shape and ultrastructural organization. Topographical imaging of the surface membrane of thrombin (0.1 U/mL)-stimulated human platelets by HR-LVSEM at 40× magnification showed induction of membrane ruffling and development of pseudopodious extensions indicative of activation (FIGS. 4A and 4B). WHI-P131 (100 μM) inhibited thrombin-induced membrane ruffling and pseudopod formation (FIGS. 4C and 4D).

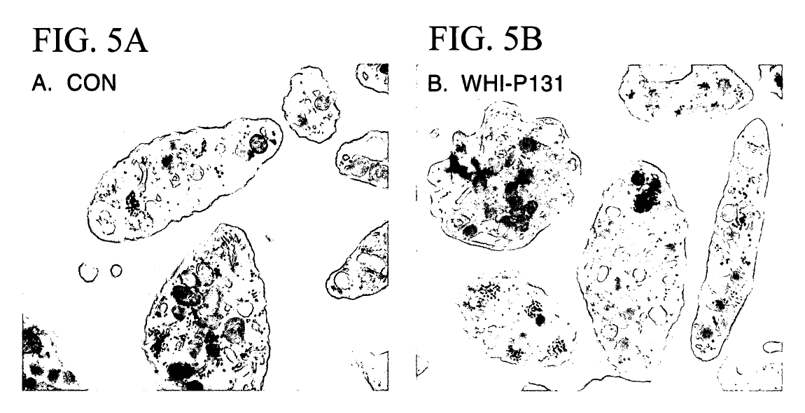

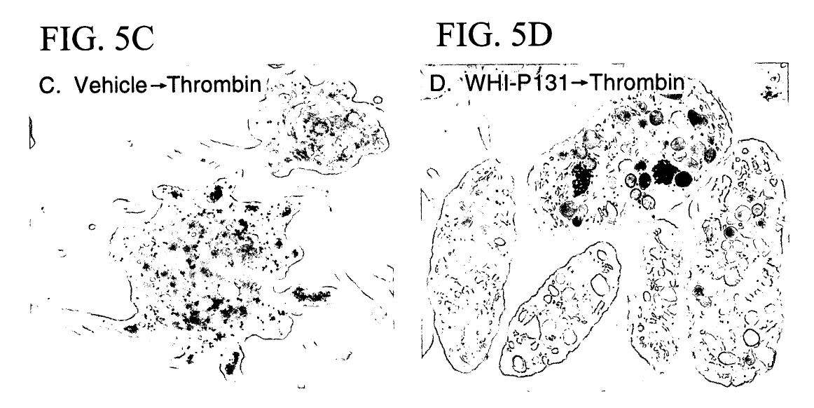

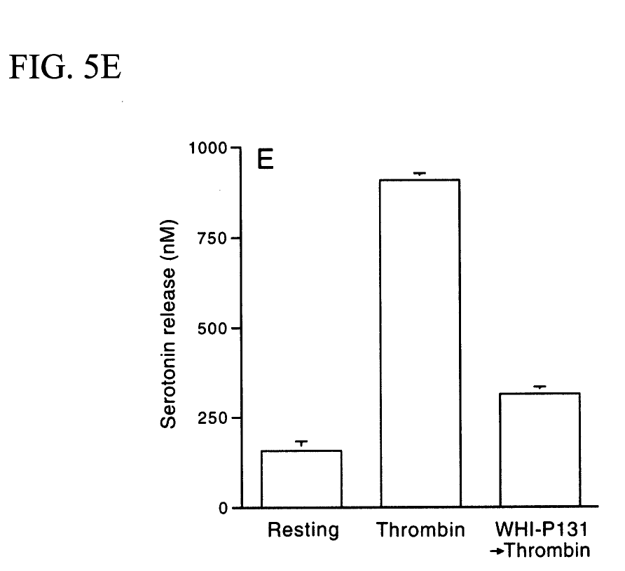

Examination of thrombin-stimulated platelets by TEM at 40,000× magnification showed a rapid shape change from discoidal cells to spheres with pseudopods extending from the surface and coalescence of granules as well as canalicular cisternae in the center of the platelet as a prelude to degranulation (FIGS. 5A and 5B). In contrast, no pseudopods were observed and the granules remained uniformly dispersed after thrombin stimulation of WHI-P131-treated platelets (FIGS. 5C and 5D).

Serotonin Release

Release of serotonin from thrombin (0.1 U/mL)-stimulated platelets was measured using a serotonin detection kit (Immunotech, Marseille, France) according to the manufacturer's specifications. Sonnicated platelets were used for measurement of the total serotonin content of platelets.

In accordance with its inhibitory effects on activation-associated shape change and granule migration in thrombin-stimulated platelets, WHI-P131 inhibited platelet degranulation after thrombin stimulation, as evidenced by a markedly reduced amount of serotonin secreted from WHI-P131-treated platelets after thrombin challenge (FIG. 5E).

The measured serotonin values in platelet supernatants were 157±26 nM (N=4) for vehicle-treated control platelets, 907±20 nM for vehicle-treated, thrombi stimulated platelets (N=4), and 313±19 (N=4) for WIH-P131 treated, thrombin stimulated platelets. Taken together, these results provide evidence that JAK3 plays critical role during the earliest events of thrombin-induced platelet activation.

### Example 3

Role of JAK3 in Thrombin-Induced Platelet Aggregation

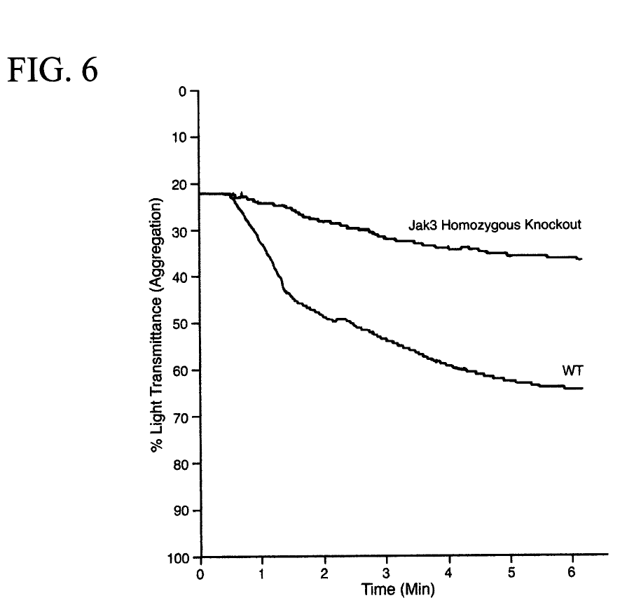

The role of JAK3 in thrombin-induced platelet aggregation was examined by first comparing the thrombin-induced aggregatory responses of platelets from wild-type and JAK3 -knockout mice, using the methods described above. As shown in FIG. 6, the magnitude of the thrombin (0.1 U/mL)-induced aggregatory response of JAK3^{+/+} platelets from wild-type mice was greater than the magnitude of the thrombin-induced aggregatory response of JAK3^{-/-} platelets from JAK3-knockout mice.

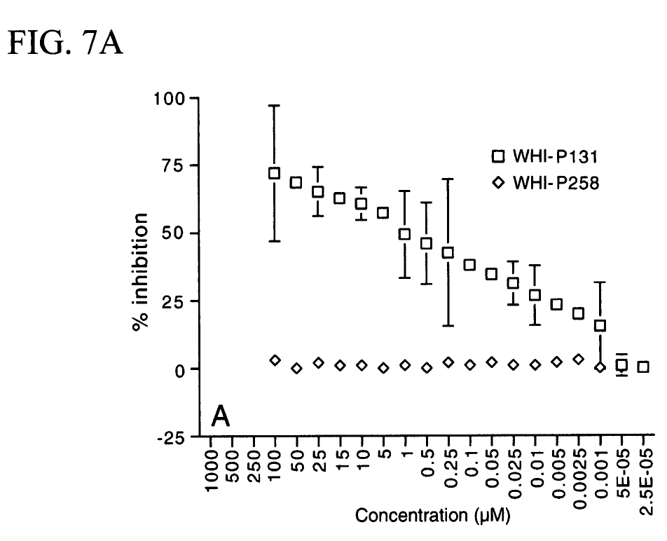

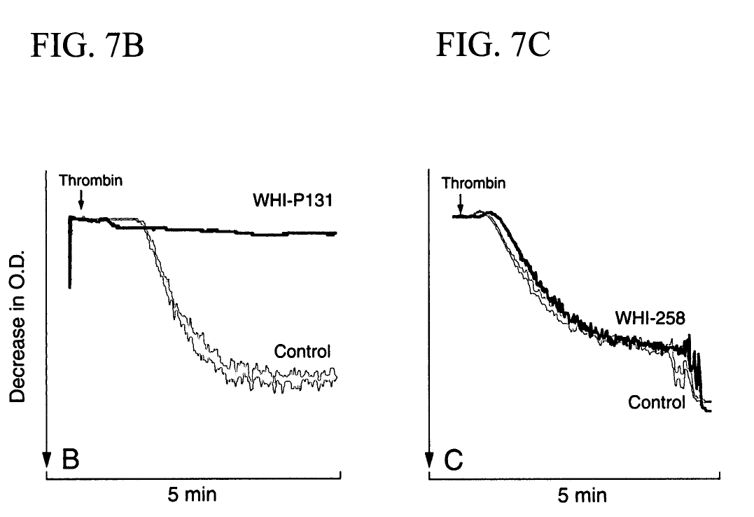

In accordance with these results, pretreatment of human platelets with the JAK3 inhibitor WHI-P131 for 30 minutes inhibited thrombin (0.1 U/mL)-induced platelet aggregation in a concentration-dependent fashion with an average IC₅₀ value of 1.5 μM (FIGS. 7A and 7B). By comparison, WHI-258, a structurally similar compound which does not inhibit JAK3, did not affect the thrombin-induced aggregation of platelets even at a 100 μM concentration (FIGS. 7A and 7C). WHI-P131 significantly reduced thrombin (FIG. 7B) but not collagen (FIG. 1D) induced platelet aggregation. WIH-P258 had no effect on the thrombin response (FIG. 7C).

### Example 4

STAT3 Isoforms

Whole cell lysates from resting platelets and FL8-2 cells (as a positive control) were collected and boiled in 2×SDS reducing sample buffer, fractionated on a 8% polyacrylamide gel and transferred to PVDF membranes. The membranes were subjected to Western blot analysis and examined for the presence of STAT-3α and STAT-3β isoforms. Both isoforms were found to be present in the platelets.

Whole cell lysates from platelets stimulated with 0.1 U/ml thrombin or 10 μg/ml collagen were collected, boiled in 2=SDS sample buffer, fractionated on an 8% polyacrylamide gel and transferred to PVDF membranes. The membranes were subjected to Western blot analysis utilizing antibodies which recognize all isoforms of STAT-3. The results show that STAT-3β tyrosine phosphorylation increased over time of thrombin stimulation, but not collagen stimulation.

Whole cell lysates from platelets treated with WHI-P131 or DMSO, stimulated with 0.1 U/ml thrombin or 10 μg/ml collagen were collected and boiled in 2×SDS sample buffer, fractionated on an 8% polyacrylamide gel and transferred to PVDF membranes. The membranes were subjected to Western blot analysis utilizing antibodies which recognize all phosphorylated isoforms of STAT-3 and phosphotyrosine. WHI-P131 inhibited thrombin induced STATU3β tyrosine phosphorylation and overall tyrosine phosphorylation.

### Example 5

WHI-P131 Prolongs Bleeding Time In Vivo and Protects Mice against Thromboplastin-Induced Fatal Thromboembolism

The effects of the JAK3 inhibitor, WHI-P131 on bleeding time and thromboplatsin-induced fatal thromboembolism were investigated using the following methods:

Measurement of Bleeding and Clotting Times in Mice

Mice (4-6 week old males, International Cancer Research (IRC)) were treated intravenously with 200 μL vehicle (PBS supplemented with 10% DMSO) or varying doses of WHI-P131 in 200 gL vehicle. To evalulate bleeding and clotting times, treated mice were placed in a tube holder and tail bleeding was performed with a 2 mm cut from the protruding tail tip; the tail was placed vertically into 10 mL normal saline in a 37° C. water bath and bleeding times determined as previously described (Teng, et.al., 1997 Eur J Pharmacol 320(2-3), 161-6).

Thromboplastin-Induced Thromboembolism Model

Mice (4-6 week old males, International Cancer Research (IRC)) were treated intravenously with 200 μL of vehicle (PBS supplemented with 10% DMSO), varying doses of WH-P131 in 200 μL of vehicle (administered intraperitoneally (i.p.) 30 minutes prior to the thromboplastin challenge). The mice were challenged with 25 mg/kg thromboplastin (Sigma, St. Louis, Mo.) via a bolus intravenous injection into the tail vein as previously described (Sato, et.al., 1998 Jpn J. Pharmacol 78(2), 191-7).

At the time of thromboembolism-related death after the thromboplastin injection or elective sacrifice at 48 hours using ketamine/xylazine, all mice were perfused with PBS followed by 4% phosphate buffered formalin. PBS and formalin were pumped through the left ventricle of the heart and allowed to exit through a 3 mm incision through the anterior wall of the right ventricle. During necropsy, several selected tissues (brain, heart, liver, lungs) were harvested, fixed in 10% neutral buffered formalin, dehydrated, and embedded in paraffin by routine methods for histopathologic examination. Glass slides with affixed 6 micron tissue sections were prepared and stained with hemotoxylin and eosin (H&E) or Masson's trichrome.

Thrombin (0.1 U/ml) induced platelet aggregation in citrated whole blood from heterozygous and homozygous JAK-3 deficient mice and C57BL/6 wild type mice was measured by optical impedence in a Model 560 Dual Chamber Chronolog Platelet Aggregometer. Platelet aggregation in response to thrombin was reduced by 65 % ∓12% in homozygous JAK-3 mice and by 17 %∓7% in heterozygous mice as compared to control.

WHI-P131 is not toxic to mice or monkeys when administered systemically at dose levels ranging from 1 mg/kg to 100 mg/kg. WHI-P131 prolonged the tail bleeding times of mice in a dose-dependent manner: the average tail bleeding times were 1.5 ±0.1 minute for vehicle-treated controls (N=12), 9.4±0.6 minute for 20 mg/kg WHI-P131 (N=5), >10 minutes for 40 mg/kg WHI-P131 (N=10), and >10 minutes for 80 mg/kg WHI-P131 (N=10).

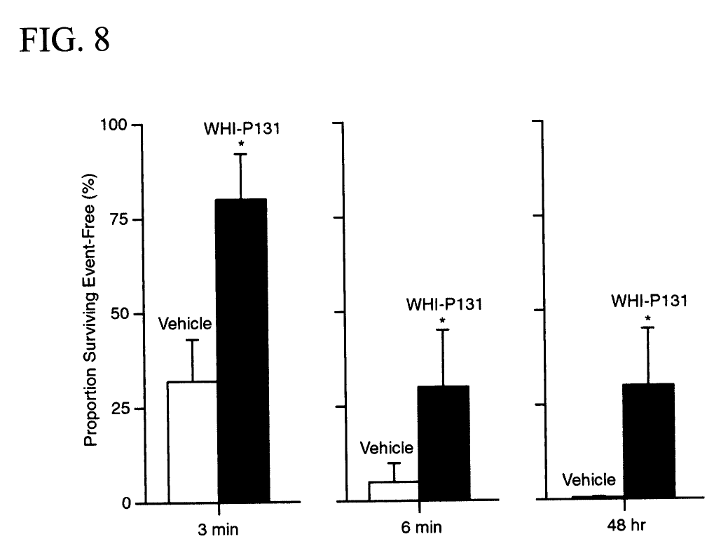

Notably, WHI-P131 also improved the survival outcome in a mouse model of thromboplastin-induced generalized and invariably fatal thromboembolism (FIG. 8). In this model, 100% of the challenged mice develop dyspnea, ataxia, and seizures and die within 10 minutes after the thromboplastin challenge from widespread thrombosis in multiple organs and massive pulmonary thromboembolism. All of the 20 vehicle-treated mice died after the thromboplastin challenge with a median survival time of 2.5 minutes.

Treatment with WHI-P131 more than doubled the median survival time and produced an event-free survival outcome of 30±15% (FIG. 8). The cause of death in WHI-P131 pretreated, thromboplastin-challenged mice was generalized thromboembolism. No drug-related toxic lesions were detected in any of the organs of these mice. All of the 20 control mice treated with 80 mg/kg WHI-P131 without a subsequent thromboplastin challenge survived beyond the 48 hour observation period without any evidence of impaired health status or bleeding.

In summary, these studies revealed an essential role for JAK3 in thrombin-induced platelet activation and aggregation. WHI-P131 inhibited thrombin-induced tyrosine phosphorylation of STAT1 and STAT3 proteins as well as activation-associated translocation of SYK and tubulin to the TX-100 insoluble fraction. In agreement with these results, platelets from JAK3 deficient mice displayed a decrease in thrombin-induced platelet aggregation and tyrosine phosphorylation of STAT 1 and STAT3. Following thrombin stimulation, WHI-P131-treated platelets did not undergo shape changes indicative of activation, such as membrane ruffling and pseudopod formation. WHI-P131 inhibited thrombin-induced degranulation/serotonin release as well as platelet aggregation.

Highly effective platelet inhibitory plasma concentrations (≧10 μM) of WHI-P131 were achieved in mice without toxicity. WHI-P131 prolonged the bleeding time of mice in dose-dependent manner and improved event-free survival in a mouse model of thromboplastin-induced generalized and fatal thromboembolism, involving the lungs, liver, heart, and CNS. Thus, the present study identifies WHI-P131 as an anti-platelet agent targeting JAK3 for prevention of potentially fatal thromboembolic events. JAK3 inhibitors such as WHI-P131 may be useful as a new class of anticoagulants for treatment of hypercoagulable metastatic cancer patients as well as patients with a primary cardiovascular, cerebrovascular, or hematologic disease at risk for thromboembolic complications.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparert to those skilled in the art from consideration of the specification and practice of the invention disclosed herein. It is intended that the specification and examples be considered as exemplary only, with a true scope and spirit of the invention being indicated by the following claims.

We claim:

1. A therapeutic method for treating or preventing a disease or condition of platelet aggregation in a subject comprising administering a pharmaceutically effective amount of a compound or composition that inhibits JAK-3.

2. The method of claim 1, wherein the compound inhibits tyrosine phosphorylation of STAT-3.

3. The method of claim 2, wherein the method selectively inhibits thrombin-induced platelet aggregation.

4. The method of claim 3, wherein the compound is represented by formula I: 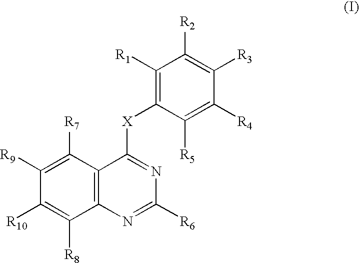
wherein:
X is selected from the group consisting of HN, R₁₁N, S, O, CH₂, and R₁₁CH;
R₁₁ is (C₁-C₄)alkyl or (C₁-C₄)alkanoyl;
R₁-R₅ are each independently selected from the group consisting of hydrogen, hydroxy, and halo where at least one of R₁-R₅ is hydroxy;
R₆, R₇, and R₈ are each independently selected from the group consisting of hydrogen, hydroxy, mercapto, amino, nitro, (C₁-C₄)alkyl, (C₁-C₄)alkoxy, (C₁-C₄)alkylthio, and halo; and
R₉ and R₁₀ are each independently selected from the group consisting of hydrogen, (C₁-C₄)alkyl, (C₁-C₄)alkoxy, halo, and (C₁-C₄)alkanoyl; or R₉ and R₁₀ together are methylenedioxy;
or a pharmaceutically acceptable salt thereof.

5. The method of claim 3, wherein the compound is represented by formula II: 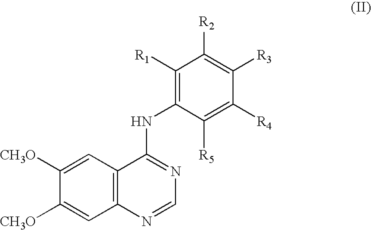
wherein:
R₁-R₅ are each independently selected from the group consisting of hydrogen, hydroxy, and halo where at least one of R₁-R₅ is hydroxy;
or a pharmaceutically acceptable salt thereof.

6. The method of claim 5, wherein the compound is 4-(4′-hydroxyphenyl)-amino-6,7-dimethoxyquinazoline; 4-(3′,5′-dibromo4′-hydroxyphenyl)-amino-6,7-dimethoxyquinazoline; 4-(3′-bromo-4′-hydroxyphenyl)-amino-6,7-dimethoxy-quinazoline; or 4-(3′-hydroxyphenyl)-amino-6,7-dimethoxyquinazoline.

7. The method of claim 6, wherein the compound is 4-(4′-hydroxyphenyl)-amino-6,7-dimethoxyquinazoline.

8. The method of claim 4, wherein the condition of platelet aggregation comprises embolus formation, thrombolytic complications, disseminated intravascular comgelopathy, thrombosis, coronary heart disease, thromboembolic complications, myocardial infarction, restenosis, or atrial thrombosis formation in atrial fibrillation.

9. A method for preventing platelet aggregation comprising administering a pharmaceutically effective amount of a compound or composition that inhibits JAK-3.
